(19) United States
(12) Patent Application Publication
(10) Pub. No.: US 2004/0208799 A1
(43) Pub. Date: Oct. 21, 2004

(54) Germicidal lamp assembly

(75) Inventor: Robert Scheir, Sherman Oaks, CA (US)

Correspondence Address: SOCAL IP LAW GROUP, 310 N. WESTLAKE BLVD. STE 120, WESTLAKE VILLAGE, CA, 91362, US

(73) Assignee: Steril-Aire USA, Inc.

(21) Appl. No.: 10/419,586

(22) Filed: Apr. 21, 2003

(51) Int. Cl. A61L 9/00; B32B 5/02

(52) U.S. Cl. 422/121; 422/24; 422/4; 422/186.3; 250/455.11

(57) ABSTRACT

There is disclosed a germicidal lamp having a ballast integrated therewith. The germicidal lamp assembly permits simpler fixture designs, and may even result in near or total elimination of the fixture. It also eases installation of a complete germicidal system (fixture plus lamp). The germicidal lamp assembly simplifies maintenance by eliminating need to separately replace the ballast and the germicidal lamp. The germicidal lamp assembly reduces product stocking requirements. Maintenance scheduling can therefore be simpler, since only the germicidal lamp assembly need be checked and/or replaced, instead of separate ballast and a germicidal lamp. The germicidal lamp assembly is compact, so it reduces warehouse and storage costs, and allows installation in smaller spaces.

A portion of the disclosure of this patent document contains material which is subject to copyright protection. This patent document may show and/or describe matter which is or may become trade dress of the owner. The copyright and trade dress owner has no objection to the facsimile reproduction by any one of the patent disclosure as it appears in the Patent and Trademark Office patent files or records, but otherwise reserves all copyright and trade dress rights whatsoever.

### BACKGROUND OF THE INVENTION

1. Field of the Invention

The present invention relates to germicidal lamps and germicidal devices that include germicidal lamps.

2. Description Of Related Art

Heating, ventilation and air conditioning (HVAC) units may be augmented with germicidal systems that emit ultraviolet light to eradicate harmful microorganisms in the air, in ducts and on and in HVAC units. Typical germicidal systems comprise a lamp and a fixture. The fixture typically includes an electronic or magnetic ballast. Germicidal systems may have one or more lamps.

### DESCRIPTION OF THE DRAWINGS

FIG. 1 is a side view of a first germicidal lamp assembly in accordance with the invention.

FIG. 2 is a partial cut-away side view of the germicidal lamp assembly of FIG. 1.

FIG. 3 is a partial elevational view of an enclosure with a germicidal lamp assembly.

FIG. 4 is a circuit diagram of a germicidal lamp assembly in accordance with the invention.

### DETAILED DESCRIPTION OF THE INVENTION

Throughout this description, the embodiments and examples shown should be considered as exemplars, rather than limitations on the apparatus and methods of the present invention.

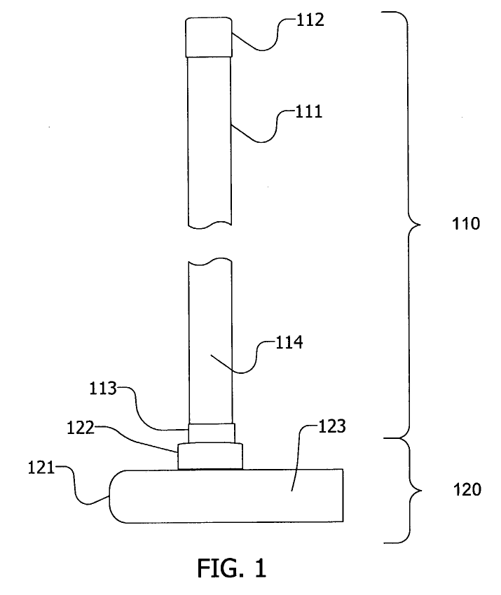

Referring now to FIG. 1 there is shown a side view of a first germicidal lamp assembly 100. The germicidal lamp assembly has two primary portions 110, 120. The first and second portions 110, 120 are structurally integrated. By “structurally integrated,” it is meant that the respective parts cannot be separated except with special tools, with special skills, or by causing structural damage.

A germicidal lamp and a ballast are included in the two portions 110, 120. The ballast may be an electronic ballast or a magnetic ballast. The germicidal lamp is adapted to produce ultraviolet light with substantially no ozone emission.

In the germicidal lamp assembly of FIG. 1, a germicidal lamp 111 is included in the first portion 110. The germicidal lamp 111 has a given effective length, such as 16″, 20″ or 41″. The germicidal lamp 111 also has a given effective output, primarily of ultraviolet light. The output of the germicidal lamp may be UV-C. The germicidal lamp may comprise a tube 114, a proximal end 113 and a distal end 112. The proximal end 113 and distal end 112 may seal gases and a material such as mercury within the tube 114. The tube 114 may be substantially clear, uncoated and made of quartz glass. The proximal end 113 is in mechanical contact with the second portion 120 of the germicidal lamp assembly 100.

The second portion 120 of the germicidal lamp assembly 100 includes a housing 121 having a main body 123 and a lamp receiving section 122. The housings 121 may be manufactured from any resilient, strong, durable material that remains functional despite temperature changes within a normal operating temperature range, such as, for example, but not limited to, metals, plastics and resins. Furthermore, the housing 121 may be sealed so that it cannot be opened except with special tools, with special skills, or by causing structural damage. The housing 121 may be sealed by means of adhesives, clips, rivets or screws, for example, and the sealing means may be a part or feature of the housing 121 or a separate component.

The lamp receiving section 122 may be adapted to receive the proximal end 113 to thereby, in part, hold the first portion 110. In the embodiment of FIG. 1, the lamp receiving section 122 is cylindrical and extends a distance from the main body 123 of the housing 121.

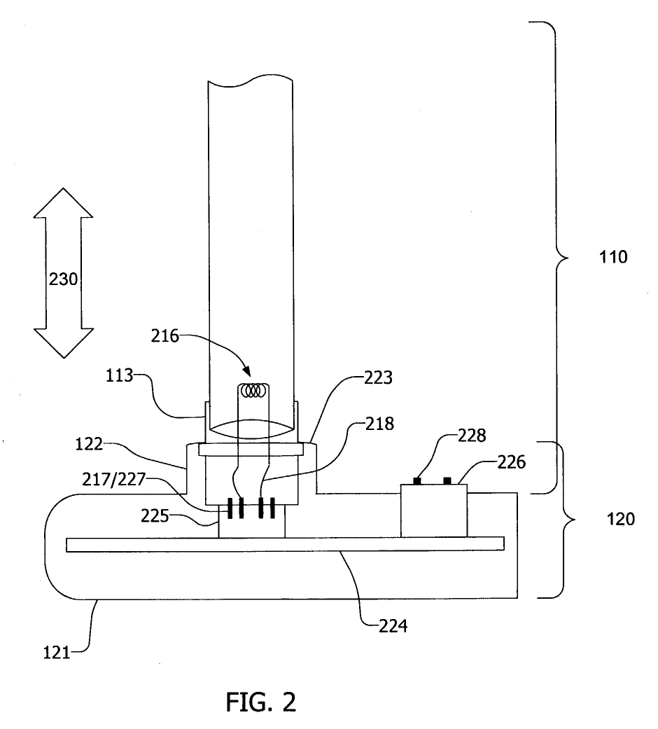

Referring now to FIG. 2, there is shown a partial cut-away side view of the germicidal lamp assembly 100 of FIG. 1. FIG. 2 provides further details of the electrical and mechanical interface between the first portion 110 and the second portion 120. The second portion 120 includes a ballast on a circuit board 224. The circuit board 224 is fixed within the housing 121.

Disposed on the circuit board 224 are a lamp connector 225 and a power connector 226. The lamp connector 225 and power connector 226 may be identical or different. The lamp connector 225 and power connector 226 provide electrical connectivity and may also provide mechanical connectivity and/or support. The lamp connector 225 and power connector 226 may be quick-connect type connectors, and may provide a locking mechanism when connected with a counterpart.

The power connector 226 provides an electrical connection to the ballast from an outside power source. The power connector 226 includes a number of electrical contacts 228. The power connector may also provide a mechanical connection to an outside structure, such as a fixture (not shown).

FIG. 2 shows that the proximal end 113 extends into the lamp receiving section 122. The proximal end 113 includes an extended portion 215 which may be in the form of a ring extending from and around the proximal end 113. The extended portion 215 presses against a lip 223 of the lamp receiving section 122. A soft, resilient material, such as a rubber gasket, may be included between the lip 223 and the extended portion 215, to thereby absorb minor shocks and provide a better fit.

In the embodiment of FIGS. 1 and 2, the first portion 110 and the second portion 120 have a mechanical connection. Forces substantially along direction 230 maintain a firm connection between the first portion 110 and the second portion 120. The housing 121 supports the circuit board 224. The circuit board 224 supports the lamp connector 225. The lamp connector 225 connects with the proximal end 113. The extended portion 215 of the proximal end 113 presses against the lip 223.

The housing 121 surrounds and thereby mechanically secures part of the proximal end 113. Accordingly, it can be seen that the first portion 110 and second portion are structurally integrated. The first portion 110 and second portion 120 may be secured to one another in many other ways without departing from the invention.

The ballast on the circuit board 224 and the germicidal lamp 111 have an electrical connection. The lamp connector 225 and germicidal lamp 111 respectively include a number of contacts 217/227. The lamp connector's contacts 227 provide the electrical connection to the ballast. The germicidal lamp's contacts 217 provide the electrical connection to the germicidal lamp 111. The proximal end 113 of the germicidal lamp 111 may include an electrode 216, as may the distal end 112. The contacts 217 may electrically connect with the electrodes. In the embodiment of FIG. 2, there are two contacts for each of two electrodes. Other arrangements are within the scope of the invention. As shown in FIG. 2, there are a pair of leads 218 from the contacts 217 to the electrode 216.

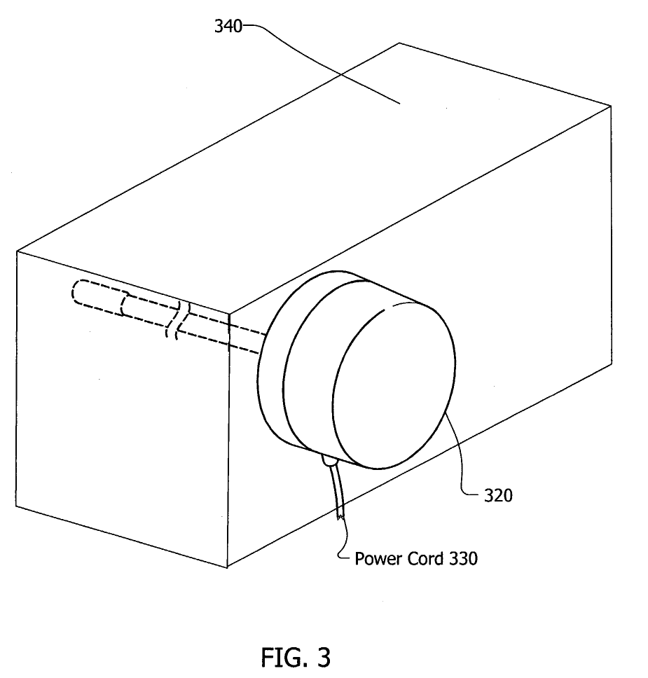

Referring now to FIG. 3 there is shown a partial elevational view of an enclosure 340 with a germicidal lamp assembly. The germicidal lamp assembly is installed in a fixture 320 to thereby form a germicidal system. The enclosure 340 may be part of an air carrying duct or an air treatment unit which includes the germicidal system. The germicidal lamp assembly of the invention is adapted for air and surface treatment.

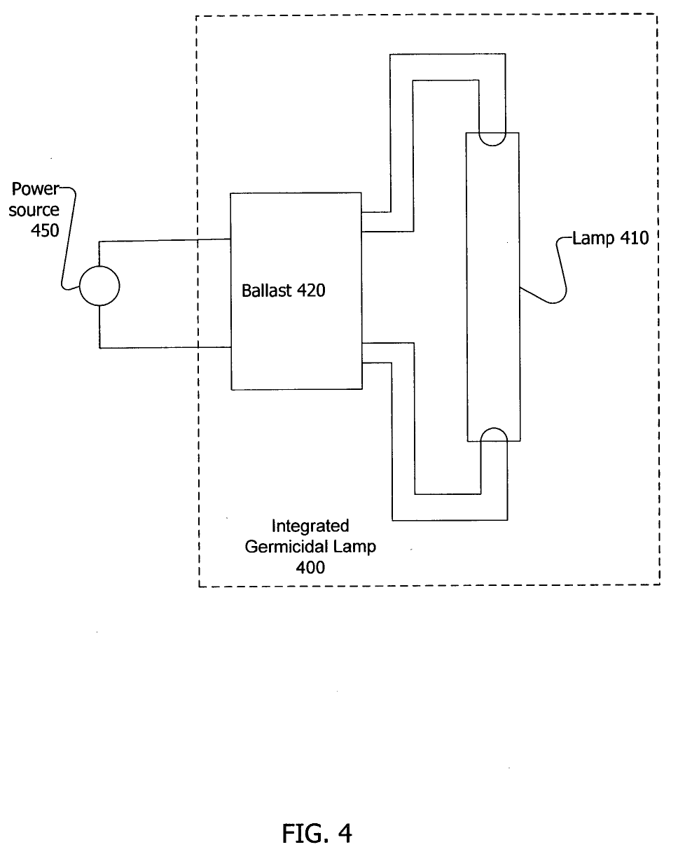

Referring now to FIG. 4 there is shown a circuit diagram of a germicidal lamp assembly 400 in accordance with the invention. The germicidal lamp assembly 400 receives power from a power source 450. The power source 450 may be AC or DC. The germicidal lamp assembly of the invention may be self-powered, for example by including one or more batteries and/or power-generating transducers.

The germicidal lamp assembly 400 includes a germicidal lamp 410 and a ballast 420. The germicidal lamp 410 has high current requirements compared to other lamps, and may draw 420 mA or 800 mA during operation, and may draw considerably more current during startup.

The ballast 420 is adapted to receive power from the power source 450 and provide power to the germicidal lamp 410. The ballast 420 itself may be physically distributed, with some significant portion included in the germicidal lamp assembly 400 and another portion not included in the germicidal lamp assembly 400. For example, some components of the ballast which have an especially long life may be outboard, while the shorter-lived components and related components may be onboard.

Both the ballast 420 and the germicidal lamp 410 have a useful life, after which their performance degrades and the germicidal lamp assembly 400 no longer produces sufficient output. The ballast 420 and germicidal lamp 410 may be adapted to have substantially the same useful life, such as one year. In this way, the cost of the germicidal lamp assembly is maximized, since neither the ballast 420 nor the germicidal lamp 410 has more quality than is necessary.

The germicidal lamp assembly of the invention may provide a number of benefits:

Permits simpler fixture designs, and may even result in near or total elimination of the fixture.

Eases installation of a complete germicidal system (fixture plus lamp).

Simplifies maintenance by eliminating need to separately replace the ballast and the germicidal lamp.

Reduces product stocking requirements.

Maintenance scheduling is simpler, since only the germicidal lamp assembly need be checked and/or replaced, instead of separate ballast and a germicidal lamp.

More compact, so reduces warehouse and storage costs, and allows installation in smaller spaces.

Because they are integrated, the ballast and germicidal lamp may be more tightly adapted to one another. This may further simplify manufacturing and maintenance, and improve quality and performance.

As a result, the germicidal lamp assembly of the invention is less expensive to manufacture, provides lower maintenance costs, and reduces repair costs.

Although exemplary embodiments of the present invention have been shown and described, it will be apparent to those having ordinary skill in the art that a number of changes, modifications, or alterations to the invention as described herein may be made, none of which depart from the spirit of the present invention. All such changes, modifications and alterations should therefore be seen as within the scope of the present invention.

It is claimed:

1. A germicidal lamp assembly for generating ultraviolet light, the germicidal lamp assembly comprising
a first portion comprising
a germicidal lamp having a given effective length and a given effective output primarily of ultraviolet light
a first plurality of contacts electrically connected to the germicidal lamp
a second portion structurally integrated with the first portion and comprising
a housing
a second plurality of contacts for mating with the first plurality of contacts
a third plurality of contacts for receiving electrical power from outside of the germicidal lamp assembly
a ballast for the germicidal lamp contained within the second portion and electrically connected to the second plurality of contacts and the third plurality of contacts.

2. The germicidal lamp assembly of claim 1 wherein the germicidal lamp comprises a tube made of quartz glass.

3. The germicidal lamp assembly of claim 1 wherein the germicidal lamp comprises a substantially clear, uncoated tube.

4. The germicidal lamp assembly of claim 1 wherein the ballast comprises an electronic ballast.

5. The germicidal lamp assembly of claim 1 wherein the ballast comprises a magnetic ballast.

6. The germicidal lamp assembly of claim 1 wherein the germicidal lamp is adapted to produce ultraviolet light with substantially no ozone emission.

7. A germicidal system comprising the germicidal lamp assembly of claim 1 and a fixture, the fixture for securing and at least partially enclosing the germicidal lamp assembly.

8. An air treatment unit including the germicidal system of claim 7.

9. An air carrying duct including the germicidal lamp assembly of claim 7.

10. A germicidal lamp assembly comprising
a germicidal lamp for treating air and surfaces
a ballast for receiving power and for providing power to the germicidal lamp
wherein the ballast is structurally integrated with the germicidal lamp.

11. The germicidal lamp assembly of claim 10 wherein the germicidal lamp comprises a tube made of quartz glass.

12. The germicidal lamp assembly of claim 10 wherein the germicidal lamp comprises a substantially clear, uncoated tube.

13. The germicidal lamp assembly of claim 10 wherein the ballast comprises an electronic ballast.

14. The germicidal lamp assembly of claim 10 wherein the ballast comprises a magnetic ballast.

15. The germicidal lamp assembly of claim 10 wherein the germicidal lamp is adapted to produce ultraviolet light with substantially no ozone emission.

16. A germicidal system comprising the germicidal lamp assembly of claim 10 and a fixture, the fixture for securing and at least partially enclosing the germicidal lamp assembly.

17. An air treatment unit including the germicidal system of claim 10.

18. An air carrying duct including the germicidal lamp assembly of claim 10.
